(19) United States
(12) Patent Application Publication
(10) Pub. No.: US 2024/0247257 A1
(43) Pub. Date: Jul. 25, 2024

(54) SYSTEM AND METHODS FOR INSERTION AND EDITING OF LARGE NUCLEIC ACID FRAGMENTS

(71) Applicant: WUHAN UNIVERSITY, Wuhan, Hubei (CN)

(72) Inventors: Hao YIN, Wuhan, Hubei (CN); Jinlin WANG, Wuhan, Hubei (CN); Ying ZHANG, Wuhan, Hubei (CN); Guoquan WANG, Wuhan, Hubei (CN); Zhou HE, Wuhan, Hubei (CN); Ruiwen ZHANG, Wuhan, Hubei (CN)

(21) Appl. No.: 18/561,669

(22) PCT Filed: May 17, 2022
(86) PCT No.: PCT/CN2022/093401

§ 371 Date: Nov. 16, 2023

(30) Foreign Application Priority Data
May 17, 2021 (WO) PCT/CN2021/094213

(51) Int. Cl. C12N 15/11; C12N 9/12; C12N 9/22; C12N 15/10

(52) U.S. Cl. CPC C12N 15/111; C12N 9/1276; C12N 9/22; C12N 15/1096; C12N 2310/20

(57) ABSTRACT

Provided are compositions and methods useful for inserting a larger nucleic acid fragment to a target genome sequence. The editing system employs a pair of pegRNA which, by virtue of their targeting nearby genomic sites and having sequences complementary to each other, collectively form a template for inserting a large exogenous sequence to the target genomic locus.

The present invention claims the priority of the PCT/CN2021/094213, filed on May 17, 2021, the contents of which are incorporated herein by its entirety.

### BACKGROUND

Targeted transgene integration is usually achieved by the homology-directed repair (HDR), which is inefficient in non-dividing cells and limited by the exogenous DNA donor. Homology-independent targeted integration (HITI) strategy has been developed to be independent of cell cycle. However, the efficiency of HITI remains low at genomic level (usually around 1-5%), and a mix of integration events was observed. Genetic deletion (including deletion/insertion) and SNP account about one fifth and two third of known human pathogenic variants, respectively. For each disease-related gene, usually a few dozen to hundreds of SNP may cause pathologic phenotype. Although a large part of SNP can be corrected by various types of base editors, practically it is difficult to develop one therapy for each SNP due to small patient populations. Alternatively, targeted inserting part of a normal gene to correct mutation by multiple types SNP is attractive. A gene editing method to achieve efficiently targeted insertion of exogenous gene with high accuracy is demanding.

A new CRISPR-based gene editor, referred to as Prime editing (PE), was recently developed through linking a reverse transcriptase (RT) to a Cas9 nickase. The RT template (RTT) is at the 3′ of the prime editing guide RNA (pegRNA), leading to precise modification of the nicked site. Prime editing is able to mediate all types of base editing, small insertion and deletion without donor DNA, holding great potential for basic research and correction of genetic mutants associated with human diseases. However, prime editing has not been used to insert larger fragment of DNA.

### SUMMARY

Efficient targeted integration holds great potential for treating a variety of genetic diseases. Current gene editing tools cannot accurately and efficiently insert exogenous genes. Prime editor can insert short fragments (˜44 bp), with limited efficiency, but cannot insert larger fragments, in part due to the requirement of the reverse transcription template (RTT) to be homologous to the target genomic sequence.

The instant inventors developed a new method, termed Grand Editing (genome editing by RT templates partially aligned to each other but non-homologous to targeted sequences duo pegRNA), that allows targeted insertion of larger fragment using pegRNA with RTT that can be non-homologous to genomic sequences. The Grand Editing employs a pair of pegRNA, neither of which requires a RT template homologous to the target genomic sequence, and thus it is not active for prime editing (prime editing requires RT template to be partial homologous to the target sequence). When used in combination, however, the dual pegRNA, by virtue of their targeting nearby genomic sites and having sequences complementary to each other, collectively form a template for inserting a large exogenous sequence to the target genomic locus. Grand Editing therefore presents a new tool for large-scale genome editing, which is useful for gene therapy and fundamental research.

One embodiment of the present disclosure provides a method for introducing a nucleic acid sequence into a target DNA sequence at a target site, comprising contacting the target DNA sequence with (a) a Cas protein and a reverse transcriptase, (b) a first prime editing guide RNA (pegRNA) comprising a first CRISPR RNA (crRNA), and a first reverse transcriptase (RT) template sequence, and (c) a second prime editing guide RNA (pegRNA) comprising a second crRNA, and a second RT template sequence, wherein (i) the first RT template sequence comprises a first fragment and a first pairing fragment, (ii) the second RT template sequence comprises a second fragment and a second pairing fragment, (iii) the first pairing fragment and the second pairing fragment are complementary to each other, (iv) the first fragment and the second fragment each has a length of 0-2000 nt, and (v) the first fragment, the first pairing fragment, and a reverse-complement of the second fragment collectively encode one of the strands of the nucleic acid sequence.

In some embodiments, the first pegRNA further comprises a first primer-binding site (PBS) and a first spacer, enabling the reverse transcriptase to reverse-transcribe the first template sequence at a first PBS target sequence near the target site that is complementary to the first PBS, and wherein the second pegRNA further comprises a second PBS and a second spacer, enabling the reverse transcriptase to reverse-transcribe the second template sequence at a second PBS target sequence near the target site that is complementary to the second PBS.

In some embodiments, the Cas protein is a nickase. In some embodiments, each pegRNA includes the first or second crRNA, the first or second pairing fragment, the first or second fragment, and the first or second PBS from 5′ to 3′ orientation.

In some embodiments, the Cas protein is a Cas12 protein. In some embodiments, each pegRNA includes the first or second crRNA, the first or second PBS, the first or second fragment, and the first or second pairing fragment, from 3′ to 5′ orientation.

In some embodiments, the reverse transcription of the first RT template sequence and the second RT template sequence results in pairing of the reverse-transcribed first pairing fragment and the reverse-transcribed second pairing fragment.

In some embodiments, the contacting occurs in the presence of a DNA repair system, which forms a double-stranded DNA sequence introduced at the target site, wherein one strand of the double-stranded DNA sequence is encoded by the first fragment, the first pairing fragment, and a reverse-complement of the second fragment collectively. In some embodiments, the target DNA sequence is in a cell, in vitro, ex vivo, or in vivo.

In some embodiments, the introduced nucleic acid sequence is least 2 bp in length, or at least 4, 20 bp, 40 bp, 60 bp, 80 bp, 100 bp, 150 bp, 200 bp, 250 bp, 300 bp, 350 bp, 400 bp, 450 bp. 500 bp, 600 bp, 700 bp, 800 bp, 900 bp, 1000 bp or 2000 bp in length.

In some embodiments, the first pairing fragment and the second paring fragment each has a length of 2-450 nt, or has a length of 4-450, 10-400, 10-300, 10-200, 10-100, 10-90, 10-80, 10-70, 10-60, 10-50, 10-40, 10-30, 20-400, 20-300, 20-200, 20-100, 20-90, 20-80, 20-70, 20-60, 20-50, 20-40, 20-30, 30-400, 30-300, 30-200, 30-100, 30-90, 30-80, 30-70, 30-60, 30-50, 30-40, 40-400, 40-300, 40-200, 40-100, 40-90, 40-80, 40-70, 40-60, 40-50, 50-400, 50-300, 50-200, 50-100, 50-90, 50-80, 50-70, 50-60, 60-400, 60-300, 60-200, 60-100, or 60-90 nt.

In some embodiments, the first fragment and the second fragment each independently has less than 95%, or less than 90%, 85%, 80%, 70%, 60%, 50%, 40%, 30%, 20%, 10% or 5%, sequence complementarity to the target DNA.

In some embodiments, the first pegRNA or the second pegRNA further comprises a tail that (a) is able to form a hairpin or loop with itself, the PBS, the RT template sequence, the crRNA, or a combination thereof, or (b) comprises a poly(A), poly(U) or poly(C) sequence, or an RNA binding domain.

In some embodiments, the nickase is a Cas9 protein containing an inactive HNH domain which cleaves the target strand. In some embodiments, the nickase is a nickase of SpyCas9, SauCas9, NmeCas9, StCas9, FnCas9, CjCas9, AnaCas9, or GeoCas9.

In some embodiments, the Cas12 protein is Cas12a, Cas12b, Cas12f or Cas12i. In some embodiments, the Cas12 protein is selected from the group consisting of AsCpf1. FnCpf1, SsCpf1, PcCpf1, BpCpf1, CmtCpf1, LiCpf1, PmCpf1. Pb3310Cpf1, Pb4417Cpf1, BsCpf1, EcCpf1, BhCas12b, AkCas12b, EbCas12b, and LsCas12b.

In some embodiments, the reverse transcriptase is M-MLV reverse transcriptase or a reverse transcriptase that can function under physiological conditions.

In some embodiments, the nickase and reverse transcriptase each is provided as a nucleotide encoding the respective protein, or as a protein.

In some embodiments, each pegRNA is provided as a recombinant DNA encoding the pegRNA, or as a RNA molecule.

Also provided, in one embodiment, is a method for introducing a nucleic acid sequence into a target DNA sequence at a target site, comprising contacting the target DNA sequence with (a) a Cas protein and a reverse transcriptase, (b) a first prime editing guide RNA (pegRNA) comprising a first crRNA, and a first reverse transcriptase (RT) template sequence, (c) a second prime editing guide RNA (pegRNA) comprising a second crRNA, and a second RT template sequence, and (d) a partially double-stranded DNA comprising a first single-stranded portion, a duplex portion, and a second single-stranded portion, wherein (i) the first single single-stranded portion has sequence homology to the first RT template sequence, and (ii) the second single-stranded portion has sequence homology to the second RT template sequence.

Another embodiment provides a method for introducing a nucleic acid sequence into a target DNA sequence at a target site, comprising contacting the target DNA sequence with (a) Cas protein and a reverse transcriptase, (b) first crRNA comprising a first spacer, (c) a first circular RNA comprising a first primer-binding site (PBS) and a first reverse transcriptase (RT) template sequence, (c) a second crRNA comprising a second spacer, and (d) a second circular RNA comprising a second PBS and a second RT template sequence, wherein (i) the first RT template sequence comprises a first fragment and a first pairing fragment, (ii) the second RT template sequence comprises a second fragment and a second pairing fragment, (iii) the first pairing fragment and the second pairing fragment are complementary to each other, (iv) the first fragment and the second fragment each has a length of 0-2000 nt. (v) the first fragment, the first pairing fragment, and a reverse-complement of the second fragment collectively encode one of the strands of the nucleic acid sequence, (vi) the PBS and the first spacer enable the reverse transcriptase to reverse-transcribe the first template sequence at a first PBS target sequence near the target site that is complementary to the first PBS, and wherein the second PBS and the second spacer enable the reverse transcriptase to reverse-transcribe the second template sequence at a second PBS target sequence near the target site that is complementary to the second PBS, and (vii) the first circular RNA and the second circular RNA are separate circular molecules or combined into a single circular molecule.

A further embodiment provides a composition or kit, comprising (a) a first prime editing guide RNA (pegRNA) comprising a first crRNA, and a first reverse transcriptase (RT) template sequence, and (b) a second prime editing guide RNA (pegRNA) comprising a second s crRNA, and a second RT template sequence, wherein (i) the first RT template comprises a first fragment and a first pairing fragment, (ii) the second RT template comprises a second fragment and a second pairing fragment, and (iii) the first pairing fragment and the second pairing fragment are complementary to each other. In some embodiments, the composition or kit further comprises a Cas protein and a reverse transcriptase.

In some embodiments, the first pairing fragment and the second paring fragment each has a length of 2-450 nt, or has a length of 10-400, 10-300, 10-200, 10-100, 10-90, 10-80, 10-70, 10-60, 10-50, 10-40, 10-30, 20-400, 20-300, 20-200, 20-100, 20-90, 20-80, 20-70, 20-60, 20-50, 20-40, 20-30, 30-400, 30-300, 30-200, 30-100, 30-90, 30-80, 30-70, 30-60, 30-50, 30-40, 40-400, 40-300, 40-200, 40-100, 40-90, 40-80, 40-70, 40-60, 40-50, 50-400, 50-300, 50-200, 50-100, 50-90, 50-80, 50-70, 50-60, 60-400, 60-300, 60-200, 60-100, or 60-90 nt.

One or more polynucleotides are provided, in some embodiments, encoding (a) a first prime editing guide RNA (pegRNA) comprising a first crRNA, and a first reverse transcriptase (RT) template sequence, and (b) a second prime editing guide RNA (pegRNA) comprising a second crRNA, and a second RT template sequence, wherein (i) the first RT template comprises a first fragment and a first pairing fragment, (ii) the second RT template comprises a second fragment and a second pairing fragment, and (iii) the first pairing fragment and the second pairing fragment are complementary to each other.

Also provided is a prime editing guide RNA (pegRNA) comprising a crRNA, a reverse transcriptase (RT) template sequence, a primer-binding site (PBS), and a tail at the 3′ side of the PBS, wherein the tail (a) is able to form a hairpin, aloop or a complex structural form with itself, the PBS, the RT template sequence, the crRNA, or a combination thereof, or (b) comprises a poly(A), poly(C), or poly(U) tail, or poly(G) sequence, or a structure/sequence recognized by RNA binding proteins. Still further provided is a method of conducting genome editing in a cell, comprising contacting the genomic DNA of the cell with a pegRNA, a Cas protein and a reverse transcriptase.

Also provided is a prime editing guide RNA (pegRNA) comprising a crRNA comprising a spacer and an RNA scaffold, fused to a first primer-binding site (PBS) and a first reverse transcriptase (RT) template sequence. Further, a method of conducting genome editing in a cell is provided, comprising contacting the genomic DNA of the cell with a pegRNA, a Cas12 protein and a reverse transcriptase. In some embodiments, the PBS and spacer enable reverse transcriptase to reverse-transcribe the RT template sequence at a target site in the genomic DNA.

### BRIEF DESCRIPTION OF THE DRAWINGS

FIG. 1. Overview of the design of GRAND editing to targeted insert DNA. Schematic representation of the paired pegRNAs to generate precise large insertion. Two Cas9 nickase-RT molecules recognize PAM sequence, bind and nick to the opposite target DNA strand, respectively. The 3′ end of nicking sites hybridize to the corresponding PBS of pegRNA, then reverse transcriptase initiates and extends the desired 3′ end-complementary new ssDNAs without homologous to genome using the RTT. Two ssDNA bind to each other through their complementary end. After equilibration between the hybridization of edited strands and original strands, the original strands are cleaved and the edited strands are repaired by gap filling and ligation.

FIG. 2. GRAND editing mediates precise large insertion at EGFP site. a, TAE agarose gel of PCR amplicons of GRAND editing-mediated 101 bp insertion with a deletion of 53 bp (as +48 bp). b, TAE agarose gel of PCR amplicons of GRAND editing-mediated 150, 200, 250, 300 and 400 bp insertions with respective deletions. The expected bands were marked with red arrows. c, The efficiencies, determined by deep sequencing of GRAND editing-mediated 101, 150, 200, 250 and 300 bp fragments insertion accompanied by a 53 bp or a 174 bp deletion. d, The editing efficiencies of 250 bp insertions at EGFP were estimated by flow cytometry. e, Insertions of 458, 600, 767 and 1085 bp determined by deep sequencing in HEK293T-EGFP cells. L. M. R: percentage of (left/middle/right mean depth/total mean depth). f, Semi-quantitative analysis of 87 bp insertion (with a 53 bp deletion) by agarose gel. g, The efficiencies of accurate insertion of short fragment and imperfect editing were measured by deep sequencing. Mean±s.d. of n=3 independent biological replicates for c-e. g.

FIG. 3. Targeted insertion of large functional fragments at EGFP site. a, Schematic showing that the 458 bp P2A-bsd gene was in-frame inserted into the EGFP locus by GRAND editing in HEK293T-EGFP cells. A representative sequence of three independent biological replicates was shown. b, The editing frequency showed in (a) was evaluated by TA cloning and subsequently Sanger sequencing of 23 individual clones. (c-f), The 315 bp EGFP coding sequence was in-frame inserted into disturbed EGFP site (341-647) to restore the function of EGFP gene (n=3 independent experiments). c, Representative images of precisely edited cells (5 days after transfection). The edited cells that restored EGFP fluorescence were pointed by white arrows. Bars, 1.000 μm. d, The edited cells with active EGFP were sorted by flow cytometry, and EGFP site was amplified and the PCR product was visualized in 1.5% agarose gel. The EGFP ctrl (line 4) was PCR product amplified from full-length EGFP plasmid. e, The GFP+ cells were sorted by flow cytometry, and the genomic DNA of each clone was for Sanger sequencing of EGFP locus. The synonymous substitutions, indicated by red stars, were designed into the inserted fragments to distinguish from the common EGFP sequences. T1 and T2: target 1 and target 2. f, The efficiency of restoring EGFP by GRAND editing was quantified by flow cytometry. Mean±s.d. of n=3 independent biological replicates.

FIG. 4. GRAND editing mediates precise large insertion at other endogenous loci. a, TAE agarose gel of PCR amplicons showing 150 bp insertion at FANCF, HEK3, PSEN1, VEGFA, LSP1 and HEK4 sites in HEK293T cells. Restriction enzyme sites are indicated by green stars, and the inserted fragment is shown in red. b, Insertion efficiency of 150 bp fragments at six endogenous loci by real-time qPCR. c, Precise insertion and imperfect editing events of 18 pairs of pegRNAs in six endogenous loci by deep sequencing. d, Insertion efficiency of 250 bp fragments at VEGFA and PSEN1 loci by real-time qPCR. Mean±s.d. of n=3-6 independent biological replicates for b&c. n=3 for d.

FIG. 5. GRAND editing mediates precise large insertion and large deletion at endogenous loci. (a-b), Insertion of 100, 150 and 200 bp fragments with various lengths of genomic DNA deletion at VEGFA and LSP1 sites in HEK293T cells. Real-time qPCR was applied to determine insertion efficiency. Mean±s.d. of n=3 independent biological replicates.

FIG. 6. Comparison of the efficiencies of accurate 150 bp insertion using GRAND editing and PE3 at five endogenous loci. a, Detection of the accurate 150 bp insertion at five sites edited by GRAND or PE3. The target regions were amplified and the PCR products were digested by HindIII restriction enzyme. The digested products were displayed by 2% TAE agarose. The digested products were indicated by the red arrows. The predicted sizes of digestion products with precise editing are listed below the image of agarose gel. b, Detecting the accurate 150 bp insertion and imperfect events of GRAND or PE3 by deep sequencing. Mean±s.d. of n=3 independent biological replicates.

FIG. 7. GRAND editing requires paired pegRNAs with partially complementary RTTs. a, Diagram of precise insertion of 3×Flag (66 bp) by paired pegRNAs. b, The accurate editing efficiencies of single 389-pegRNA, 433-pegRNA or paired pegRNAs treated samples were determined by deep sequencing. c, Diagram of paired pegRNAs (pegA and pegB) with/without complementary region to insert fragment into genome. d, Deep sequencing of paired pegRNAs without partial complementary RTT to each other. e, The editing efficiencies by 10, 20, 40, 60, 80 or 100 bp complementary ends to insert 100, 200 and 250 bp at EGFP (268-433) site were quantified by deep sequencing. f-g, DNA fragments of 100, 150, 200 and 250 bp were inserted at VEGFA-4 site and EGFP (341-433) site with different lengths of complementary base pairs. The editing efficiencies were measured by real-time qPCR (f) and FACS (g). Mean±s.d. of n=3 independent biological replicates for b, d, e-g.

FIG. 8. Paired pegRNAs with no homology to genome outperformed pegRNAs with homologous RTT sequences. a, Overview of three design schemes to insert 66 bp 3×Flag fragment. b, Sanger sequencing confirmed the editing outcomes of the three design schemes of pegRNAs. The purple arrow indicates the installed point mutation. c, Estimated insertion efficiencies of three design schemes by deep sequencing. d, Diagram of 20 bp insertion with or without deletion. e, Comparison of the accurate editing efficiencies of two strategies shown in (d). Mean±s.d. of n=3 independent biological replicates for c and e.

FIG. 9. Paired pegRNAs with fully active Cas9 nuclease-reverse transcriptase (aPE) mainly induced deletion between two double stranded breaks. a, The diagram indicates the editing outcome of fully active Cas9 nuclease version of GRAND editing (aPE). b, Insertion of 87 or 101 bp using GRAND editing or aPE. The editing outcomes were measured by TAE agarose gel (n=3 independent experiments). c, The Sanger sequencing result of aPE completely aligned with WT sequence with a 53 bp deletion between two double stranded breaks. d, Insertion of 150 bp foreign DNA fragments accompanied with deletion of genomic DNA by GRAND editing or aPE. The target sites were amplified using primers that bound to adjacent genomic regions. The expected precise editing bands were pointed by red arrows. e, All of the edited bands were purified by gel electrophoresis, and deep sequencing analysis was performed. Mean±s.d. of n=3 independent biological replicates expects VEGFA-del 348 bp in aPE.

FIG. 10. GRAND editing mediates precise large insertion in various cell lines. Targeted insertion of a 150 bp fragment in K562 cells, Huh-7 cells and N2a cells at various loci. Efficiencies of insertion were determined by real-time qPCR. Mean±s.d. of n=3 independent biological replicates.

FIG. 11. GRAND editing mediates precise large insertion in non-dividing cells. a, The proliferation of RPE cells at 6 h, 12 h, 24 h, 48 h after treated with 1 or 2.5 μM Palbociclib, or 100, 200, 400 ng/mL Nocodazole was determined by cell counting. b, The cell cycles of RPE cells were determined by propidium iodide staining. c, Detection of the nascent DNA synthesis in RPE cells by 5-ethynyl-2′-deoxyuridine (EdU) incorporation assay. The proportions of EdU-labeled positive cells were determined by flow cytometry. d, A 100 bp DNA fragment was inserted at EGFP (595-647) site in non-dividing RPE cells using GRAND editing. The accurate editing and imperfect events were quantified by deep sequencing. Mean±s.d. of n=3 independent biological replicates for a, b, d, and n=3-5 independent biological replicates for c.

FIG. 12. Haripin-pegRNA (hp-pegRNA) improves the editing efficiency of prime editing. a, The design strategy of different types of hp-pegRNA. b, Comparing the editing efficiency of wt-pegRNA and hp-pegRNA in HEK293T-cGFP cells targeting EGFP gene. c, hp-pegRNA (R5-R) has higher editing efficiency in 10 endogenous gene loci in HEK293T cells and N2A cells compared with WT-pegRNA.

FIG. 13. Poly-A tail element significantly improves PE2 and PE3 editing efficiency in large editing window. a, Schematic of poly-A tail strategy. The poly-A tail is added to the 3′ end of PBS. (b-c), PegRNA with 100-nt RT included 4 mutations in 89-nt editing window. Sanger sequencing results show the editing efficiency of PE2 or PE3 system with or without poly-A tail element. d, PegRNA with 200-nt RT included 6 mutations in 190-nt editing window. Sanger sequencing results show that combining PE3 with Poly-A tail element can greatly increase the editing efficiency.

FIG. 14. Combining PE2-paired pegRNAs system with a pegRNA structure looping (SL) can further improve the efficiency of large insertion. a. The SL is located at the 3′ end of PBS. and it's complementary to the 5′ end of RT. b, Inserting different lengths of fragments using Grand editing system to disrupt the expression of EGFP by gene insertion. Left: A representative flow cytometry analysis shows the different editing efficiency with or without SL. Right: Estimated insertion efficiencies of different lengths of fragments by flow cytometry.

FIG. 15. Overview of Cas12 nucleases-mediated prime editing. The Cas9 nickase in the classical Prime Editor system is replaced by the Cas12 nuclease, plus the corresponding pegRNA consists of crRNA. RTT, and PBS. It is noteworthy that the RTT and PBS are located at the 5′ end of the crRNA, as 5′-RTT-PBS-crRNA-3′ (This composition is distinct different from pegRNA for Cas9: 5′-sgRNA-RTT-PBS-3′. The mechanisms of the new Cas12-PE system are as follows: (1) The Cas12 nuclease, fused to a reverse transcriptase, assembles with special pegRNA into a complex (5′-RTT-PBS-crRNA-3′). (2) The Cas12-PE complex binds and cleaves its target DNA to form staggered ends. (3) Edited ssDNA is reversely transcribed by RT enzyme using RTT template. And the RTT sequence contains the interest edit which is marked with an asterisk. (4) The edit strands compete with the original strands, and when the edit strands are complementary with the genome, a 5′ flap occurs. (5) After cellular 5′ flaps cleavage and DNA repair, the original DNA is replaced with the edited DNA.

FIG. 16. Overview of Cas12 nucleases-mediated GRAND editing. Schematic representation of the special dual-pegRNAs derived from crRNAs to replace the original pegRNAs in Grand editing to generate precise large insertion. Two Cas12 nuclease-RT: pegRNA complexes recognize PAM sequence, bind and cleave to form staggered ends, respectively. The new ssDNAs polymerized by reverse transcriptase annealing to each other with complementary 3′ ends. After equilibration between the hybridization of edited strands and original strands. the original strands are cleaved and the edited strands are repaired by gap filling and ligation.

FIG. 17. Schematic of an optimized version of the Grand Editing (GEmax) architecture. The dual-pegRNAs in classical Grand editing consist of a traditional pegRNA structure made up of sgRNA and a 3′ extended sequence. The optimized version splits dual pegRNAs into two single sgRNAs and one or more circRNAs, and the circRNA contains RTT and PBS sequences.

FIG. 18. Overview of a derivative version of Grand editing (dvGE) mediates targeted insertion and feasibility studies in 293T cells. a, Schematic of a derivative version of Grand editing mediates target insertion. Two Cas9 nickase-RT:pegRNA complexes bind and nick the target DNA, then two ssDNA are generated by reverse transcriptase using RTTs. The two ssDNAs have no complementary region with each other and genomic DNA. Therefore, when there is no donor, the genome will be restored to the original state, and when there is a donor provided, the donor will hybridize with two new ssDNAs resulting in the insertion of an exogenous DNA sequence. b, The table reflects the specific design details of the 10 dsDNA donors. c, Editing efficiency of 10 kinds of dsDNA donors targeted insert in VEGFA-4 site. Mean±s.d. of n=2 independent biological replicates.

FIG. 19. Diversity of donor designs in the dvGE. Two Cas9 nickase-RT:pegRNA complexes acting on the target DNA causes two 3′ flaps without complementary regions. When the donor is provided, the flap A in genome will hybridize with flap a in donor and the flap B will hybridize with flap b in donor. Based on this premise, donors can be provided in a variety of ways as follow: (1) dsDNA with 3′ overhang as a donor; (2) donor is available in the form of plasmid or minicircle DNA, the flap in the donor can be generated by Prime Editor; (3) based on (2), two nick sites provided by nickase:sgRNA complexes are downstream of the 2 flaps' sites; (4) differently from (2), flap a and flap b are generated by Cas nuclease-RT rather than Cas nickase-RT.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein”, “amino acid chain” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “Cas protein” or “clustered regularly interspaced short palindromic repeats (CRISPR)-associated (Cas) protein” refers to RNA-guided DNA endonuclease enzymes associated with the CRISPR (Clustered Regularly Interspaced Short Palindromic Repeats) adaptive immunity system in Streptococcus pyogenes, as well as other bacteria. Cas proteins include Cas9 proteins, Cas12a (Cpf1) proteins, Cas12b (formerly known as C2c1) proteins, Cas13 proteins and various engineered counterparts. Example Cas proteins include SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EcCpf1, BhCas12b, AkCas12b, EbCas12b, LsCas12b, RfCas13d, LwaCas13a, PspCas13b, PguCas13b, RanCas13b.

### Grand Editing

The present disclosure provides a new genetic editing method, termed Grand Editing (genome editing by RT templates partially aligned to each other but non-homologous to targeted sequences duo pegRNA), that enables insertion or replacement of nucleic acid fragments to target genomic sequences.

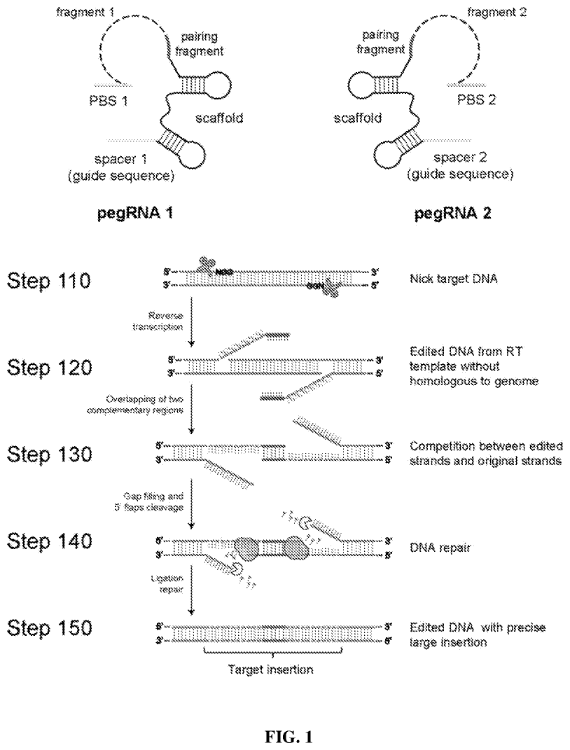

An example Grand Editing process employs a pair of prime editing guide RNA (pegRNA) molecules illustrated in FIG. 1. A conventional pegRNA includes, in addition to a CRISPR RNA (crRNA) (which can be provided, along with a trRNA, as a single guide RNA (sgRNA)), a reverse transcriptase (RT) template sequence and a primer binding site (PBS). The PBS is complementary to the guide sequence (or “spacer”) in the sgRNA, but is typically a few nucleotides shorter. When the guide sequence binds to the target genome sequence and dissociates the DNA double helix, the PBS binds to the opposite strand and initiates reverse transcription, using the RT template sequence as a template. The RT template can include mutations or small insertions relative to the target genome sequence, but needs to be largely homologous to the target genome sequence.

In each of the two pegRNA of a Grand Editing system, the RT template does not have to be homologous to the target genome sequence. In some embodiments, the RT template preferably has reduced or even no homology to the target genome sequence. Instead, the two RT templates share a complementary portion. For instance, as illustrated in FIG. 1, in the first pegRNA (pegRNA 1), the RT template includes two portions, a pairing fragment and a fragment 1; in the second pegRNA (pegRNA 2), the RT template also includes two portions, a pairing fragment and a fragment 2. The two pairing fragments have complementary sequences (or substantially complementary, such as at least 40%, 60%, 70%, 80%, 90% or 95% complementary sequence identity) so that they can pair with each other.

The pairing does not need to occur between the two pegRNA molecules. Instead, upon binding to the target genome sequence (Step 110), both pegRNA will serve as templates to generate (by reverse transcription) DNA sequences (single-stranded) (Step 120). As the lower panel of FIG. 1 shows, by virtue of the complementary sequences and their close proximity, these two newly reverse transcribed single-stranded DNA fragments can bind to each other at their respective 3′ ends (Step 130). The non-paired portions (reverse transcribed from RT template of pegRNA 1 and RT template of pegRNA 2) can then serve as template for DNA replication, generating a double-stranded DNA sequence encoded collectively by fragment 1, the pairing fragment, and fragment 2 (reverse complement) (Step 150). Accordingly, a DNA fragment encoded collectively by the two pegRNA is inserted between the two nicking sites. Meanwhile, if there is an existing fragment between the two nicking sites in the genome, it will be replaced by this newly inserted fragment. The Grant Editing method, therefore, can replace existing genomic sequences or insert new sequences.

A significant advantage of the Grant Editing technology is that it can insert very large fragments into a genome. For instance, if each RT template (fragment 1 or 2+pairing fragment) is 1000 nucleotides in length, then the total length of the inserted fragment is about 2000 nucleotides.

The lower end of the insertion or replacement size can be small too. If both fragment 1 and fragment 2 are zero in length (non-existent), the minimum length of the pairing fragment can be 2 nucleotides to enable pairing, then the total length is just 2 bp.

Another advantage is that neither fragment 1 nor fragment 2 nor pairing fragments needs to be homologous to the target genomic sequence, as required by prime editing. Therefore, the Grand Editing can be employed to insert any sequences.

Yet another advantage is the increased editing specificity and efficiency. Given that Grant Editing requires two pegRNA each has guide sequences, and thus the editing can only happen at genomic loci having complementary sequences to both guide sequences, the specificity is necessarily improved. Further, as demonstrated in the experimental examples, the editing efficiency is many folds higher than prime editing. Also, as Grand Editing does not rely on cells' DNA repair function to remove unedited DNA strands, it is more reliable and independent.

Moreover, as discussed below, the present disclosure further discloses improved pegRNA designs which not only increase prime editing efficiency but also further improves Grand Editing.

Accordingly, one embodiment of the present disclosure provides a method for introducing a nucleic acid sequence into a target DNA sequence at a target site. In some embodiments, the method entails contacting the target DNA sequence with (a) Cas protein (e.g., a regular Cas9, Cas12 or Cas13 protein, or a nickase) and a reverse transcriptase (optionally combined in a fusion protein, or separately provided), (b) a first prime editing guide RNA (pegRNA) comprising a first single guide RNA (sgRNA) (or alternatively just a crRNA), and a first reverse transcriptase (RT) template sequence, and (c) a second prime editing guide RNA (pegRNA) comprising a second single guide RNA (sgRNA) (or alternatively just a crRNA), and a second RT template sequence. In some embodiments, the first RT template includes a first fragment and a first pairing fragment, the second RT template includes a second fragment and a second pairing fragment, and the first pairing fragment and the second pairing fragment are complementary to each other. The pairing fragment can be in the middle, or at either 3′ or 5′ end of the fragment 1 (a first fragment) or 2 (a second fragment).

Collectively, the first fragment, the first pairing fragment, and a reverse-complement of the second fragment encode one of the strands of the nucleic acid sequence. It is noted that the first fragment and the second fragment each can be empty (0 nucleotide), or can be as long as thousands of nucleotides.

The pegRNA disclosed herein can include other elements of conventional pegRNA as used in prime editing.

Prime editing is a genome editing technology by which the genome of living organisms may be modified. Prime editing directly writes new genetic information into a targeted DNA site. It uses a fusion protein, consisting of a catalytically impaired endonuclease (e.g., Cas9) fused to an engineered reverse transcriptase enzyme, and a prime editing guide RNA (pegRNA), capable of identifying the target site and providing the new genetic information to replace the target DNA nucleotides. Prime editing mediates targeted insertions, deletions, and base-to-base conversions without the need for double strand breaks (DSBs) or donor DNA templates.

The pegRNA is capable of identifying the target nucleotide sequence to be edited, and encodes new genetic information that replaces the targeted sequence. The pegRNA consists of an extended single guide RNA (sgRNA) (or alternatively just a crRNA) containing a primer binding site (PBS) and a reverse transcriptase (RT) template sequence. During genome editing, the primer binding site allows the 3′ end of the nicked DNA strand to hybridize to the pegRNA, while the RT template serves as a template for the synthesis of edited genetic information. Within the sgRNA or crRNA portion, there are a spacer (guide sequence) that guides the prime editor to the target genomic site, and a sgRNA/crRNA scaffold.

The fusion protein, in some embodiments, includes a nickase fused to a reverse transcriptase. A nickase can be derived from a regular Cas9 protein, such as SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, or CjCas9. An example nickase is Cas9 H840A. The Cas9 enzyme contains two nuclease domains that can cleave DNA sequences, a RuvC domain that cleaves the non-target strand and a HNH domain that cleaves the target strand. The introduction of a H840A substitution in Cas9, through which the histidine residue at 840 is replaced by an alanine, inactivates the HNH domain. With only the RuvC functioning domain, the catalytically impaired Cas9 introduces a single strand nick, hence a nickase.

Non-limiting examples of reverse-transcriptases include human immunodeficiency virus (HIV) reverse-transcriptase, moloney murine leukemia virus (M-MLV) reverse-transcriptase and avian myeloblastosis virus (AMV) reverse-transcriptase, and any reverse transcriptases that can function under physiological conditions.

In some embodiments, the prime editing system further includes a single guide RNA (sgRNA) (or alternatively just a crRNA) that directs the Cas9 H840A nickase portion of the fusion protein to nick the non-edited DNA strand. It is noted, however, that such an extra sgRNA/crRNA is not required in the Grand Editing system.

Prime editing can be carried out by transfecting target cells with the pegRNA and the fusion protein. Transfection is often accomplished by introducing vectors into a cell. In some embodiments, the prime editors can be introduced to a cell directly as plasmids, linear DNA, proteins, RNA, and virus-like particles, or their complexes. Each molecule can be introduced separately, or together, without limitation.

Vectors may be introduced into the desired host cells by known methods, including, but not limited to, transfection, transduction, cell fusion, and lipofection. Vectors can include various regulatory elements including promoters. In some embodiments, the present disclosure provides an expression vector including any of the polynucleotides described herein, e.g., an expression vector including polynucleotides encoding the fusion protein and/or the pegRNA.

The spacers and the PBS can be designed such that they bind to genomic sequences flanking a region wherein DNA insertion and/or replacement is desired.

Accordingly, in some embodiments, the first pegRNA further includes a first primer-binding site (PBS) and a first spacer, enabling the fusion protein or complex to reverse-transcribe the first template sequence at a first PBS target sequence near the target site that is complementary to the first PBS, and the second pegRNA further includes a second PBS and a second spacer, enabling the fusion protein or complex to reverse-transcribe the second template sequence at a second PBS target sequence near the target site that is complementary to the second PBS. In some embodiments, the reverse transcription of the first RT template sequence and the second RT template sequence results in pairing of the reverse-transcribed first pairing fragment and the reverse-transcribed second pairing fragment.

In some embodiments, the contacting occurs in the presence of a DNA repair system, which forms a double-stranded DNA sequence introduced at the target site, wherein one strand of the double-stranded DNA sequence is encoded by the first fragment, the first pairing fragment, and a reverse-complement of the second fragment collectively. Such contacting can be, for instance, in a cell, in vitro, ex vivo, or in vivo. The cell may be a prokaryotic cell, a eukaryotic cell, a plant cell, an animal cell, a mammal cell, or a human cell.

The introduced nucleic acid sequence, whether for insertion only or insertion and replacement, is at least 2 bp in length. Preferably, however, the length of the inserted or replaced sequence is at least 45 bp in length, or at least 60 bp, 80 bp, 100 bp, 150 bp, 200 bp, 250 bp, 300 bp, 350 bp, 400 bp, 450 bp, 500 bp, 600 bp, 700 bp, 800 bp, 900 bp, 1000 bp or 2000 bp in length.

The first and second pairing fragments just need to be long and homologous enough to enable their sequences to pair. In some embodiments, each of them has a length of 2-450 nt, or has a length of 4-450, 10-400, 10-300, 10-200, 10-100, 10-90, 10-80, 10-70, 10-60, 10-50, 10-40, 10-30, 20-400, 20-300, 20-200, 20-100, 20-90, 20-80, 20-70, 20-60, 20-50, 20-40, 20-30, 30-400, 30-300, 30-200, 30-100, 30-90, 30-80, 30-70, 30-60, 30-50, 30-40, 40-400, 40-300, 40-200, 40-100, 40-90, 40-80, 40-70, 40-60, 40-50, 50-400, 50-300, 50-200, 50-100, 50-90, 50-80, 50-70, 50-60, 60-400, 60-300, 60-200, 60-100, or 60-90 nt.

As disclosed, the first fragment and the second fragment do not need to be homologous to the genomic sequences to be replaced. In some embodiments, the first fragment and the second fragment each independently has less than 95%, or less than 90%, 85%, 80%, 70%, 60%, 50%, 40%, 30%, 20%, 10% or 5%, sequence complementarity to the target DNA.

Also provided are compositions, kits and packages useful for conducting Grand Editing. In some embodiments, the composition, kit or package includes at least a pair of pegRNA useful for the editing, as described herein.

In some embodiments, the pair of pegRNA include (a) a first prime editing guide RNA (pegRNA) comprising a first single guide RNA (sgRNA) (or alternatively just a crRNA), and a first reverse transcriptase (RT) template sequence, and (b) a second prime editing guide RNA (pegRNA) comprising a second single guide RNA (sgRNA) (or alternatively just a crRNA), and a second RT template sequence. In some embodiments, the first RT template comprises a first fragment and a first pairing fragment, (ii) the second RT template comprises a second fragment and a second pairing fragment, and (iii) the first pairing fragment and the second pairing fragment are complementary to each other.

Further included in the composition, kit or package may be a fusion protein or complex comprising a nickase and a reverse transcriptase.

In some embodiments, the composition, kit or package includes polynucleotide (e.g., DNA) sequences that encode the two pegRNA disclosed herein. The DNA sequences can be provided in a single sequence or a single vector, or in separate sequences or vectors, without limitation. The fusion protein or complex can also be provided as encoding polynucleotide sequences, in some embodiments.

The first fragment, one of the pairing fragments, and the second fragment (the reserve complement thereof) collectively encode a nucleic acid sequence to be inserted to a target genome sequence. In some embodiments, the encoded sequence is at least 2 bp in length. Preferably, however, the length of the inserted or replaced sequence is at least 45 bp in length, or at least 60 bp, 80 bp, 100 bp, 150 bp, 200 bp, 250 bp, 300 bp, 350 bp, 400 bp, 450 bp, 500 bp, 600 bp. 700 bp. 800 bp. 900 bp. 1000 bp or 2000 bp in length.

The first and second pairing fragments just need to be long and homologous enough to enable their sequences to pair. In some embodiments, each of them has a length of 2-450 nt, or has a length of 10-400, 10-300, 10-200, 10-100, 10-90, 10-80, 10-70, 10-60, 10-50, 10-40, 10-30, 20-400, 20-300, 20-200, 20-100, 20-90, 20-80, 20-70, 20-60, 20-50, 20-40, 20-30, 30-400, 30-300, 30-200, 30-100, 30-90, 30-80, 30-70, 30-60, 30-50, 30-40, 40-400, 40-300, 40-200, 40-100, 40-90, 40-80, 40-70, 40-60, 40-50, 50-400, 50-300, 50-200, 50-100, 50-90, 50-80, 50-70, 50-60, 60-400, 60-300, 60-200, 60-100, or 60-90 nt.

Improved pegRNA Molecules

Example 2 demonstrates the construction and testing of three new pegRNA structures, all of which exhibited greater editing efficiency when used for prime editing and/or Grand editing.

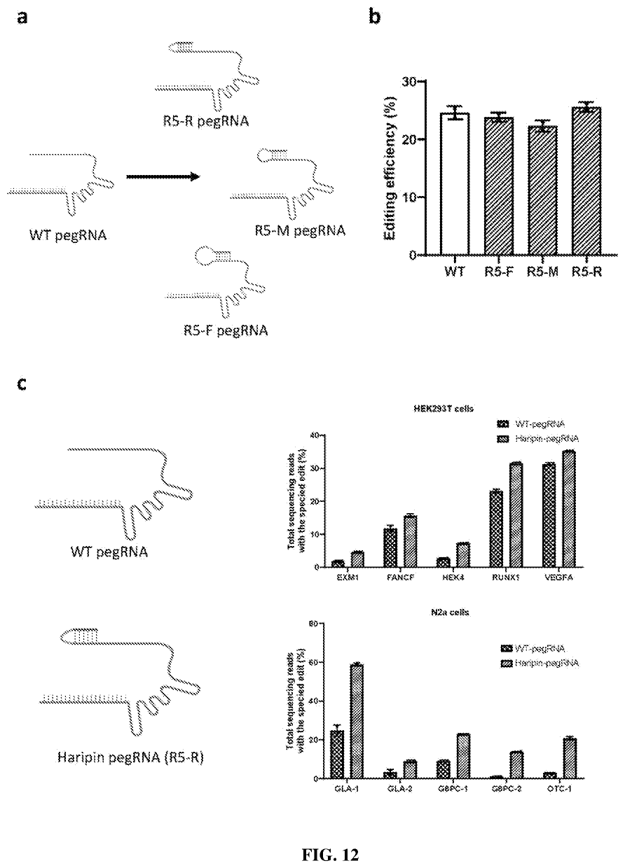

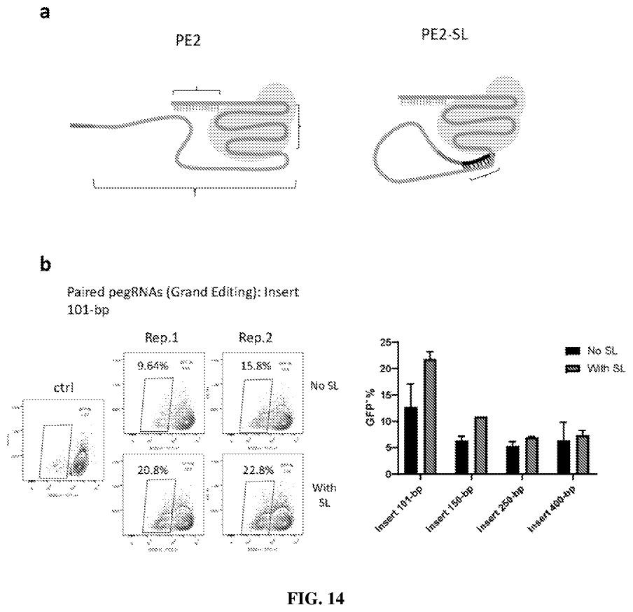

A first design is illustrated in FIG. 12, in which a tail is introduced to the 3′ end of the pegRNA that is able to form a hairpin with PBS or the RT template. Similarly, in the third design (FIG. 14), the tail binds to the PBS, the RT template, or the sgRNA/crRNA scaffold to form a loop. Either the hairpin or the loop helps stabilizes the pegRNA. Moreover, the hairpin or loops reduces the interaction between the PBS (in the hairpin or loop) and the complementary guide sequence (spacer), ensuring that the guide sequence functions effectively to bind to the target editing site.

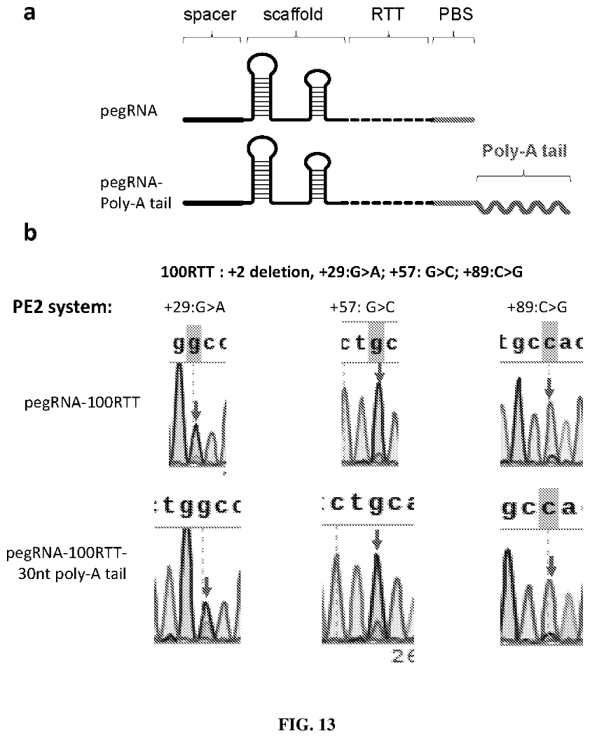

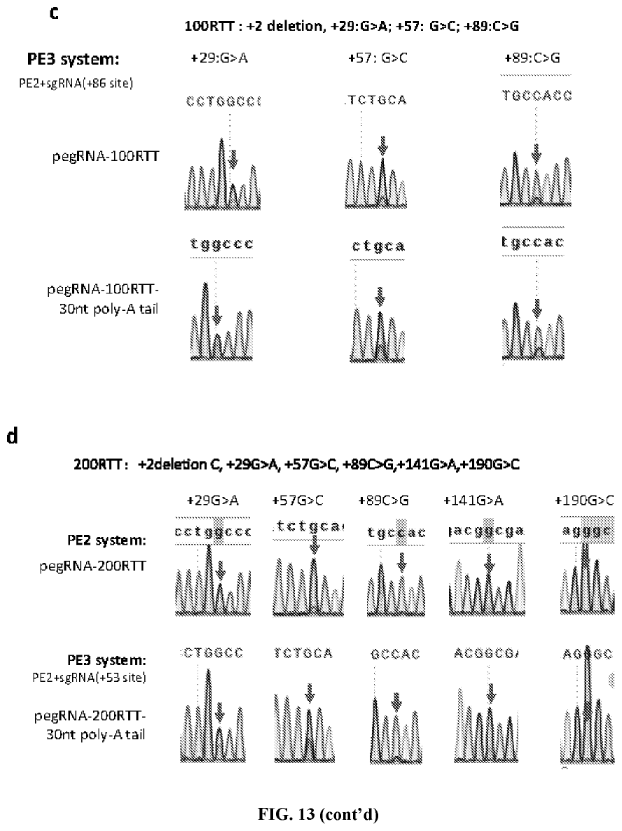

The second design is illustrated in FIG. 13, in which a poly(A) tail is appended at the 3′ end of a conventional pegRNA. All of these designs resulted in increased editing efficiency which, to some extent, was unexpected. This is at least because it was suspected that the added sequences may reduce speed of degradation of pegRNA.

Accordingly, one embodiment of the present disclosure provides a prime editing guide RNA (pegRNA) comprising a single guide RNA (sgRNA) (or alternatively just a crRNA), a reverse transcriptase (RT) template sequence, a primer-binding site (PBS), and a tail. In some embodiments, the tail is at the 3′ side of the PBS. In some embodiments, the tail is at the 3′ end of the pegRNA.

In some embodiments, the tail is able to form a hairpin with itself, with the PBS, or with the RT template. In some embodiments, the tail is able to form a loop by binding to the PBS, the RT template sequence, the sgRNA/crRNA (e.g., the scaffold), or a combination thereof. In some embodiments, the tail has a length of at least 4 nucleotides, or at least 5, 6, 7, 8, 9, 10, 11, 12, 13, 14, 15, 20, 25, or 30 nt. In some embodiments, the tail is not longer than 100 nt, or not longer than 90, 80, 70, 60, 50, 40, 30, 20, 10 or 5 nt.

In some embodiments, the tail comprises a poly(A) sequence. In some embodiments, the poly(A) has a length of at least 4 nucleotides, or at least 5, 6, 7, 8, 9, 10, 11, 12, 13, 14, 15, 20, 25, or 30 nt. In some embodiments, the tail or poly(A) is not longer than 100 nt, or not longer than 90, 80, 70, 60, 50, 40, 30, 20, 10 or 5 nt.

In some embodiments, the tail can comprise a poly(A), poly(U), poly(C), poly(G) or other polynucleotide sequence. In some embodiments, the tail includes an intrachain base-pairing or folding of the ribonucleotide chain into complex structural forms such as bulges and helices or other three-dimensional structures. In some embodiments, the tail at the 3′ end of the pegRNA includes poly (A) tail, poly(C) tail, poly(U) tail, poly(G) tail, random polynucleotides tail, separately, or together.

In some embodiments, a pegRNA can include one or more chemical modifications. Example nucleic acid chemical modifications include N6-methyladenosine (m6A), inosine (I), 5-methylcytosine (m5C), pseudouridine (Ψ), 5-hydroxymethylcytosine (hm5C), N1-methyladenosine (m1A), Phosphorodithioate (PS), boranophosphate (BP), 2′-O-methoxyethyl (2′-MOE), locked nucleic acids (LNA), unlocked nucleic acids (UNA), 2′-deoxy, 2′-O-methyl (2′-OMe), 2′ fluoro (2′-F), 2′-methoxyethyl, 2′-aminoethyl, 2′ thiouridine. In some embodiments, the proportion of chemical modifications on pegRNA accounts for 5%, or 10%, 20%, 30%, 40%, 50%, 60%, 70%, 80%, 90%, 100%.

These improved pegRNA structures can be used for both the conventional prime editing systems and in the currently disclosed Grand editing systems, without limitation.

Also provided are methods of using the improved pegRNA for genome editing, and prime or Grand editing compositions, kits and packages for genome editing.

### Cas12-Based Prime Editing and Grant Editing

The conventional PE2 system is composed of Cas9 nickase-RT and pegRNA. The Cas12 proteins, however, have not been used in prime editing, primarily due to the lack of a corresponding Cas12 nickase. The conventional pegRNA is not expected to work with Cas12. A Cas9 nickase introduces a single-strand cut, but a Cas12 protein cuts both strands. A conventional pegRNA includes a single guide RNA (sgRNA) (or alternatively just a crRNA) which includes a spacer and a scaffold, a reverse transcriptase (RT) template sequence and a primer binding site (PBS), in a spacer-scaffold-RTT-PBS (5′ to 3′) configuration. If the target genome is cut in both strands by the Cas12 protein, the RTT in the pegRNA cannot serve as an effective RT template.

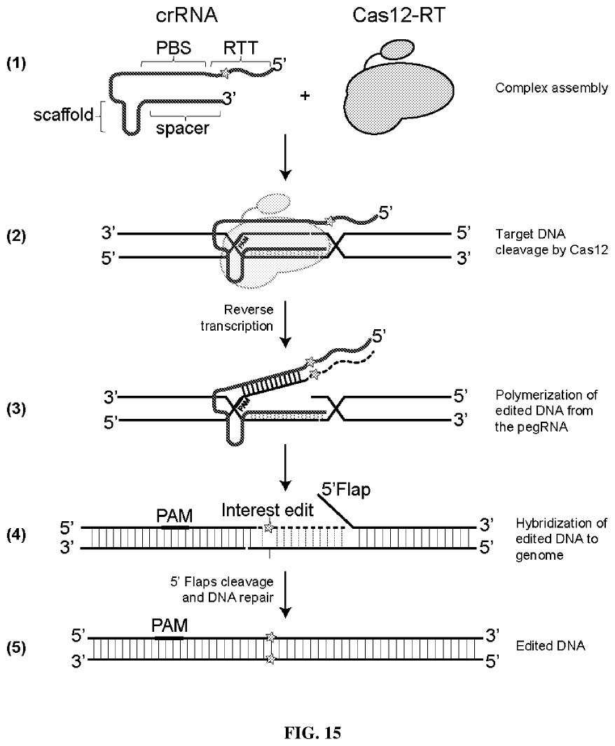

One embodiment of the present disclosure provides a prime editing system based on Cas12, which is illustrated in FIG. 15. Instead of adopting the spacer-scaffold-RTT-PBS (5′ to 3′) configuration of a conventional pegRNA, the new pegRNA has a RTT-PBS-scaffold-spacer (5′ to 3′) configuration. In other words, in this new pegRNA, the PBS and RTT are located at the 5′ side of the crRNA scaffold (hereafter referred to as cr-pegRNA). As illustrated in FIG. 15, despite the double strand cuts made by the Cas12 protein, the Cas12-based primer editing system is able to insert a fragment complementary to the RTT, which can optionally include a desired mutation (“interest edit”).

The new cr-pegRNA structure also has the advantage in protecting PBS from exonuclease digestion. For RTT, it can slow down the degradation by adding a secondary structure or extending the length of RTT. This special arrangement of elements may greatly improve the stability of pegRNA, thereby improving the editing efficiency of Prime Editing. In addition, the shorter length of the crRNA means that the length of the cr-pegRNA will also be greatly shortened than pegRNA. Therefore, cr-pegRNA has great advantages in industrial synthesis of modified pegRNA.

Using Cas12 nuclease may generate a staggered end on genome which is different from the blunt end caused by Cas9 or nick caused by nCas9. In addition, as compared to nCas9, a fully-active Cas12 may have higher cleavage activity and less dependency on special sites and contexts.

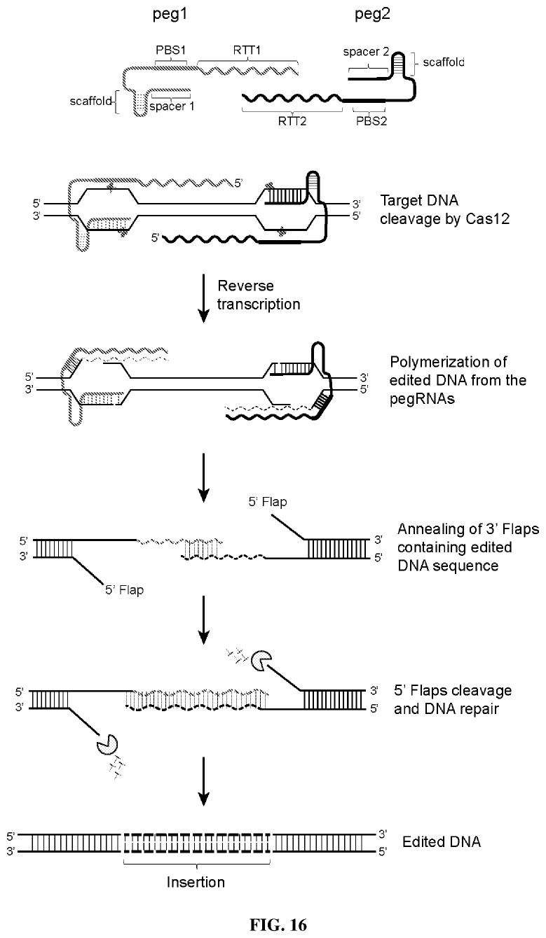

The newly developed Cas12/cr-pegRNA system can also be used in Grand Editing. One such implementation is illustrated in FIG. 16. Different from original design of Grand editing (FIG. 1), the nCas9-RT is replaced with Cas12-RT, and the dual-pegRNAs are replaced with dual-(cr-pegRNA)s including complementary regions in the RTTs. Same as original Grand editing, the two new ssDNAs anneal with each other using the complementary regions and the 5′ flaps are cleaved by endogenous exonuclease. After DNA repair, the foreign DNA is targeted insertion into the genome. It is worth noting that Cas12 can generate staggered ends which can benefit DNA repair preferring edited DNA. Therefore, this new system can insert and/or delete short or long sequences in genome.

Accordingly, in one embodiment, provided is a method for introducing a nucleic acid sequence into a target DNA sequence at a target site, comprising contacting the target DNA sequence with (a) a fusion protein or complex comprising a Cas protein and a reverse transcriptase, (b) a first prime editing guide RNA (pegRNA) comprising a first single guide RNA (sgRNA) (or alternatively just a crRNA), and a first reverse transcriptase (RT) template sequence, and (c) a second prime editing guide RNA (pegRNA) comprising a second single guide RNA (sgRNA) (or alternatively just a crRNA), and a second RT template sequence, wherein (i) the first RT template sequence comprises a first fragment and a first pairing fragment, (ii) the second RT template sequence comprises a second fragment and a second pairing fragment, (iii) the first pairing fragment and the second pairing fragment are complementary to each other, (iv) the first fragment and the second fragment each has a length of 0-2000 nt, and (v) the first fragment, the first pairing fragment, and a reverse-complement of the second fragment collectively encode one of the strands of the nucleic acid sequence.

The Cas protein may be a Cas12 protein, which may be Cas12a, Cas12b, Cas12f and Cas12i, without limitation. Examples include AsCpf1. FnCpf1, SsCpf1, PcCpf1. BpCpf1. CmtCpf1, LiCpf1, PmCpf1, Pb3310Cpf1, Pb4417Cpf1, BsCpf1, EcCpf1, BhCas12b, AkCas12b, EbCas12b, and LsCas12b.

In some embodiments, each pegRNA includes the first or second spacer, the first or second sgRNA (or alternatively just a crRNA), the first or second PBS, the first or second fragment, and the first or second pairing fragment, from 3′ to 5′ orientation.

It is appreciated that various embodiments described above for nickase are applicable for the Cas12-based Grand Editing systems as well including, for instance, preferred length of the nucleic acid elements, without limitation.

In some embodiments, a pegRNA is provided, comprising a single guide RNA (sgRNA) (or alternatively just a crRNA) comprising a spacer and an RNA scaffold, fused to a first primer-binding site (PBS) and a first reverse transcriptase (RT) template sequence. Also provided is a method of conducting genome editing in a cell, comprising contacting the genomic DNA of the cell with a pegRNA, and a fusion protein or complex comprising a Cas12 protein and a reverse transcriptase.

In some embodiments, the PBS and spacer enable the fusion protein or complex to reverse-transcribe the RT template sequence at a target site in the genomic DNA.

Split pegRNA and cr-pegRNA

The present disclosure, in some embodiments, provides new configurations and delivery mechanisms for pegRNA and cr-pegRNA, including those for basic prime editing and for Grand Editing. In one embodiment, a pegRNA (or likewise for a cr-pegRNA) is split into two RNA molecules.

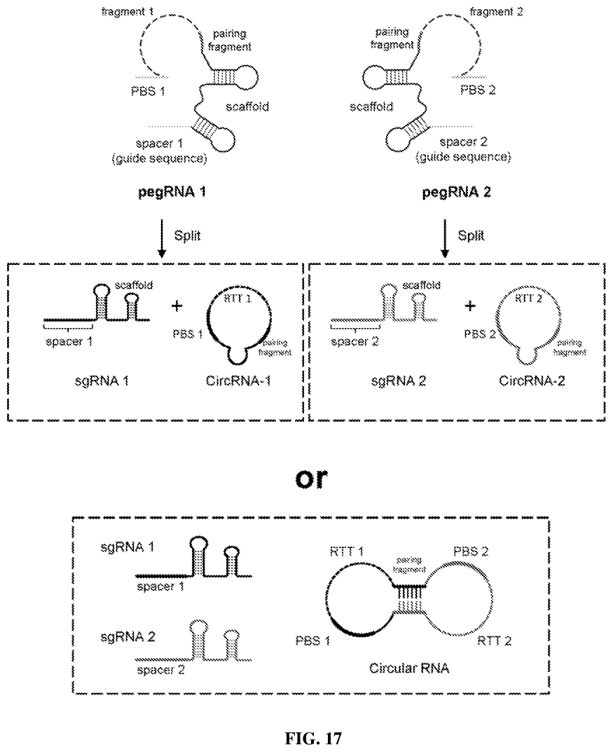

As illustrated in FIG. 17, in one embodiment, the PBS and RTT portions can be provided as a circular RNA molecule, separately from the sgRNA (or alternatively just a crRNA) portion. As both the spacer of the sgRNA (or alternatively just a crRNA) and the PBS in the circular RNA can recognize the target genomic site, they can be brought together by virtue of such recognition.

It should be appreciated that such configurations are generally applicable to pegRNA for any prime editing system. In some implementations, this configuration is specifically applied to Grand Editing. In one example, both pegRNA (or both cr-pegRNA) molecules are provided as split molecules (upper panel in FIG. 17). In some embodiments, the two circular RNA molecules are provided in a unified one (lower panel in FIG. 17) which can further stabilize the RNA molecules, in particular because the two “pairing fragments” can form a double-stranded portion. Grand Editing with such split pegRNA molecules are hereby referred to as GEmax.

Therefore, one embodiment provides a method for introducing a nucleic acid sequence into a target DNA sequence at a target site, comprising contacting the target DNA sequence with one or more of (a) a fusion protein or complex comprising a Cas protein and a reverse transcriptase, (b) first single guide RNA (sgRNA) (or alternatively just a crRNA) comprising a first spacer, (c) a first circular RNA comprising a first primer-binding site (PBS) and a first reverse transcriptase (RT) template sequence, (c) a second single guide RNA (sgRNA) (or alternatively just a crRNA) comprising a second spacer, and (d) a second circular RNA comprising a second PBS and a second RT template sequence.

In some embodiments, (i) the first RT template sequence comprises a first fragment and a first pairing fragment. In some embodiments, (ii) the second RT template sequence comprises a second fragment and a second pairing fragment. In some embodiments, (iii) the first pairing fragment and the second pairing fragment are complementary to each other. In some embodiments, (iv) the first fragment and the second fragment each has a length of 0-2000 nt. In some embodiments, (v) the first fragment, the first pairing fragment, and a reverse-complement of the second fragment collectively encode one of the strands of the nucleic acid sequence. In some embodiments, (vi) the PBS and the first spacer enable the fusion protein or complex to reverse-transcribe the first template sequence at a first PBS target sequence near the target site that is complementary to the first PBS, and wherein the second PBS and the second spacer enable the fusion protein or complex to reverse-transcribe the second template sequence at a second PBS target sequence near the target site that is complementary to the second PBS. In some embodiments, (vii) the first circular RNA and the second circular RNA are separate circular molecules or combined into a single circular molecule.

### Bridged Grand Editing

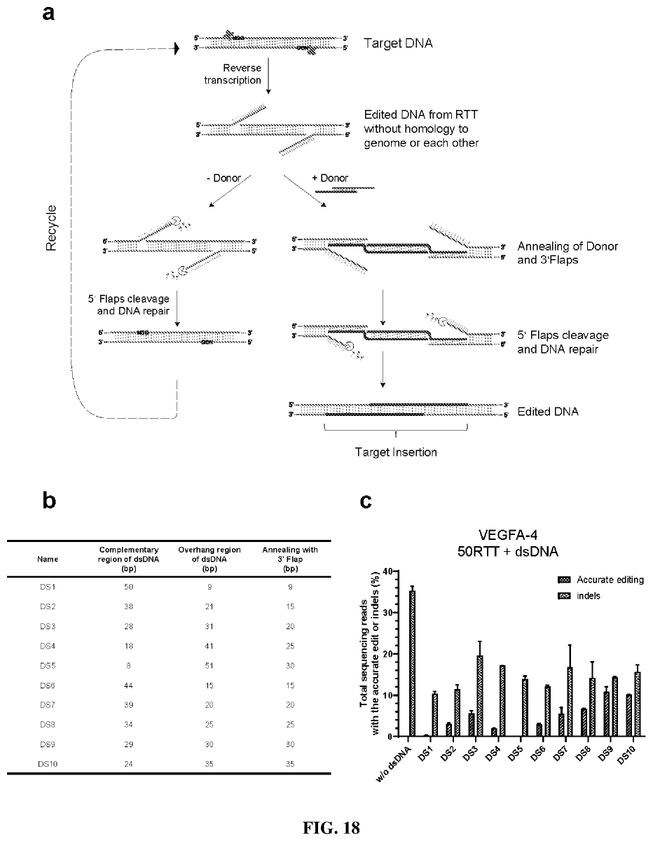

An alternative design for the Grand Editing technology is also provided, in some embodiments. In the implementation illustrated in FIG. 1, the two pegRNA molecules each includes a “pairing fragment” within the RTT, which are complementary to each other. In an alternative implementation illustrated in FIG. 18, the two new ssDNAs polymerized from RT have no complementary regions with each other. Therefore, the damaged genome may restore its original state when there is no donor. However, when a suitable donor (bridging, partially double-stranded DNA) is provided, the ssDNAs can hybridize with the donor to form a relatively stable structure and finally result in the desired DNA modification.

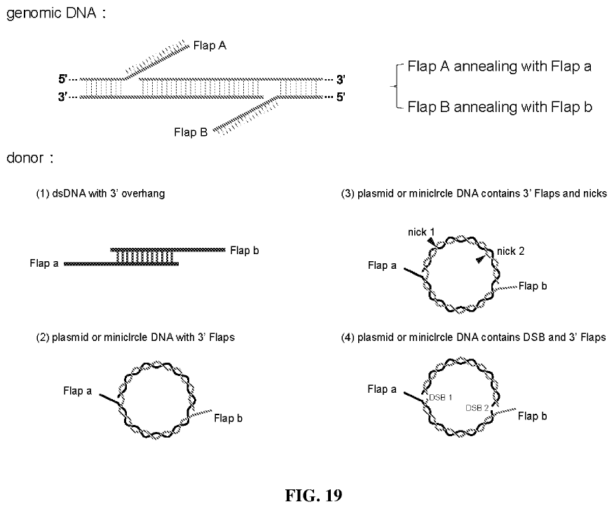

Example designs of the donor are illustrated in FIG. 19. The first design structure is a simple dsDNA with two 3′ overhangs, and the overhangs contain the sequences which are complementary to flaps in genome. The second design structure is a plasmid or a minicircle DNA with reasonable 3′ flaps generated from prime editor in cells. The third design structure contains two flaps and two nicks. Based on the second design structure, two nicks are generated near the flaps on the plasmid or minicircle DNA donors, which is in order to facilitate the dsDNA containing 3′ flaps free from the cyclized structure. The fourth design structure is generated from prime editor with full-active Cas nuclease. And the double-strand breaks (DSB) on the plasmid or minicircle DNA donors make the dsDNA containing 3′ flaps easy to release. Generally, compare with the first design structure, the latter three donor design structures all have higher stability and relatively lower cytotoxicity.

Accordingly, one embodiment provides a method for introducing a nucleic acid sequence into a target DNA sequence at a target site, comprising contacting the target DNA sequence with (a) a fusion protein or complex comprising a nickase and a reverse transcriptase, (b) a first prime editing guide RNA (pegRNA) comprising a first single guide RNA (sgRNA) (or alternatively just a crRNA), and a first reverse transcriptase (RT) template sequence, (c) a second prime editing guide RNA (pegRNA) comprising a second single guide RNA (sgRNA) (or alternatively just a crRNA), and a second RT template sequence, and (d) a partially double-stranded DNA comprising a first single-stranded portion, a duplex portion, and a second single-stranded portion, wherein (i) the first single single-stranded portion has sequence homology (e.g., sufficient sequence identity (e.g., >50%, 60%, 70%, 80%, 90%, 95% or 98%) to allow hybridization of one to the other's complement) to the first RT template sequence, and (ii) the second single-stranded portion has sequence homology to the second RT template sequence.

### EXAMPLES

### Example 1. Development and Testing of Grand Editing

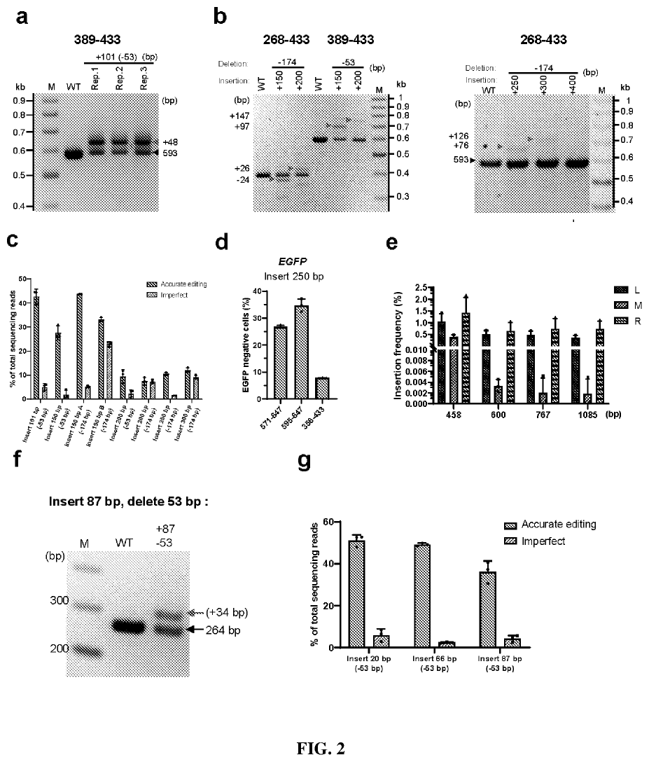

In this example, we developed a method named Grand editing (genome editing by RT templates partially aligned to each other but non-homologous to targeted sequences duo pegRNA), to precisely insert larger fragment of DNA, ranged from 20 bp to ˜1 kp. The efficiency of targeted insertion is high, about 66.0% for targeted insertion of ˜100 bp, ˜44.9% for 150 bp, ˜28.4% for 200 bp, ˜27.0% for 250 bp and ˜12.1% for 300 bp (FIG. 6f, FIG. 2c).

To prevent the cleavage of newly transcribed DNA and introduce the formation of 5′ Flap, the pegRNA of PE system must have a RTT which can hybrid to targeted region. We contemplated that a pair of pegRNA of which 3′ end is complimentary to each other, can hybrid to each other to prevent formation of 3′ Flap, thereby these pegRNA may not need homologous RTT for targeted insertion (FIG. 1, lower panel). We first designed a pair of pegRNA, aiming to insert 101 bp fragment at EGFP site in HEK293T cells with integrated EGFP gene (HEK293T-EGFP). The RTT of this paired pegRNA had 40 bp complementary sequences to each other at 3′ end, and both RTTs have no homology to the genomic sequences. We predicted that this strategy would insert 101 bp, and meanwhile delete the sequences (53 bp) between 2 nicks caused by Cas9 nickase. PCR amplification of target region showed a band of original size and a +48 bp band (101−53=48 bp). The band intensity suggests the insertion rate was efficient considering the PCR bias towards shorter fragments (FIG. 2a).

We named this method of targeted insertion as Grand Editing, and used it to insert 150 bp, 200 bp. 250 bp, 300 bp and 400 bp size DNA fragments, respectively (these sequences are part of Firefly Luciferase gene). Gel electrophoresis showed bands of all predicted sizes except for 400 bp insertion at EGFP site (FIG. 2b). To analyze editing accuracy, we sequenced PCR products by amplicon sequencing and found that GRAND editing mediated 42.7% of accurate editing of total events for 101 bp insertion (FIG. 2c). We tested different pairs of pegRNAs for 150 bp or 200 bp insertion. The efficiencies of accurate editing varied from 43.7% for 150 bp insertion to 7.6% for 200 bp insertion (FIG. 2c). For 250 bp and 300 bp insertion the efficiencies of accurate editing were 10.5% and 12.1%, respectively (FIG. 2c). For 101 bp insertion, 5.1% of total genomic sequences were imperfect editing (FIG. 2c). We noticed that if the RTT sequence contains microhomology with the target sequence such as samples of Insert 150 bp B. Insert 200 bp, and Insert 300 bp, the ratios of imperfect insertion were substantial (FIG. 2c). We thus codon optimized the RTT to avoid microhomology with the target site, and this optimization significantly reduced the imperfect editing from 23.0% (Insertion 150 bp B) to 5.1% (Insertion 150 bp A), and accurate editing increased from 33.1% to 43.7% (FIG. 2c). When designing RTT, it is important to avoid microhomology between each RTT and target site as well as between two RTTs other than the complementary end. We examined three additional pairs of pegRNAs in EGFP locus for insertion of 250 bp to explore whether higher editing efficiency could be reached. Due to the potential PCR bias between the inserted genotype and unedited genotype, we used flow cytometry analysis to estimate the knock-in efficiency for EGFP locus. It showed that 7.8% to 34.8% EGFP negative cells were generated by these pairs of pegRNAs, suggesting efficient knock-in to destroy EGFP reading frame (FIG. 2d).

To investigate the ability to insert 400 bp or larger, a 458 bp P2A-bsd gene (Blasticidin S deaminase), and DNA fragments of 600 bp, 767 bp and ˜1 kb (1085 bp) were designed to insert into the EGFP site using GRAND editing. Deep sequencing analysis revealed that the efficiency of targeted insertion of 458 bp was 0.38% (without drug-induced enrichment), and the efficiencies for 600 bp, 767 bp and ˜1 kb insertion were 0.003%, 0.002% and 0.002%, respectively (FIG. 2e). Of note, the portion of partial insertion was higher than perfect insertion for 458 bp and larger size of insertion (FIG. 2e). Due to the potential bias introduced by PCR, the efficiencies of larger insertion may be severely underestimated. Further studies are needed to improve the efficiency of perfect insertion of 400 bp to 1 kb DNA fragment.

We also examined whether GRAND editing could insert fragments shorter than 101 bp, such as 87, 66 and 20 bp. Deep sequencing analysis showed efficiencies ranged from 36.2% to 51.1% for insertion of short fragments, accompanied by deletion of a 53 bp sequence between two nicking sites (FIG. 2f-g).

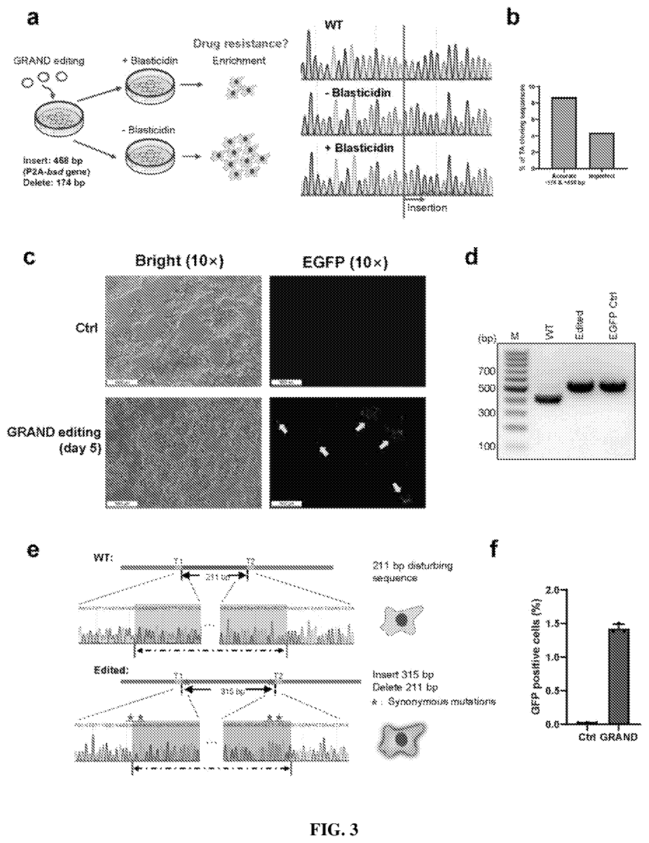

To investigate whether the 458 bp bsd gene is functional after insertion, blasticidin was added to test the activity of Blasticidin S deaminase. Eight days post treatment, cells were harvested for DNA Sanger sequencing analysis. Successful enrichment was confirmed by Sanger sequencing to demonstrate blasticidin resistance (FIG. 3a-b).

To explore whether GRAND editing could repair a “broken” gene, we generated a “broken” EGFP in which a 315 bp sequence was replaced with a 211 bp random sequence. We applied GRAND editing to insert the 315 bp sequence and delete the 211 bp random sequence (FIG. 3c-f). Five days after transfection, EGFP positive cells were observed under fluorescence microscope, while control group (PE2 plasmid only) showed no EGFP positive cells (FIG. 3c). Flow cytometry analysis demonstrated 1.4% cells were EGFP positive (FIG. 3f). Gel electrophoresis and Sanger sequencing further confirmed the precise modifications in EGFP positive cells (FIG. 3e).

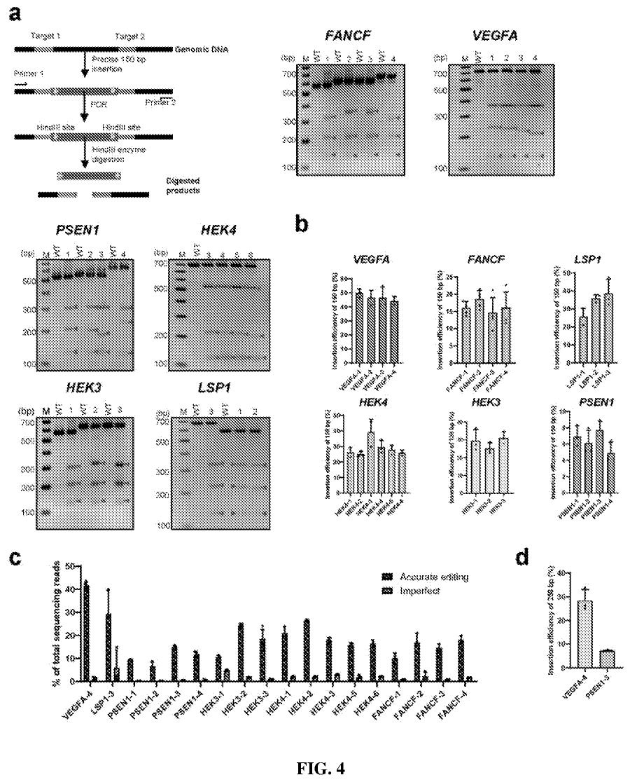

We further expanded GRAND editing to modify other endogenous sites in human genome, including FANCF, HEK3, PSEN1, VEGFA, LSP1 and HEK4. For each site, 3-6 pairs of pegRNAs were tested, and a total of 24 pairs were examined for GRAND editing. These pairs of pegRNAs contain the same RTT to insert a 150 bp fragment containing two HindIII digestion sites (FIG. 4a). Amplicons were with HindIII endonuclease, and all paired pegRNAs treated samples exhibited cut bands of expected sizes, indicating correct insertion by GRAND editing (FIG. 4a).

To determine the accurate insertion rate, we developed real-time qPCR assay by designing primers flanking junction sites and selected pairs of primers with similar amplification curve to calculate copy numbers. We found that the insertion rate of 150 bp sequence ranged from 44.2% to 50.0% for VEGFA site, 14.7% to 18.6% for FANCF site, 25.7% to 38.6% for LSP1 site, 25.0% to 39.2% for HEK4 site, 25.1% to 31.2% for HEK3 site and 4.9% to 7.7% for PSEN1 site depending on the pegRNAs (FIG. 4b).

Deep sequencing analysis of amplicon estimated the accurate editing sequences to be 6.5% to 41.7% with a minor portions of imperfect editing events (FIG. 4c). Although there were some variations in efficiencies determined by real-time qPCR and amplicon sequencing, these approaches collectively demonstrated the activities of GRNAD editing.

Furthermore, we inserted 250 bp fragment into VEGFA and PSEN1 sites to showcase that GRAND editing can insert fragments larger than 150 bp at endogenous sites. Measured by real-time qPCR, the insertion efficiencies for VEGFA and PSEN1 were 28.4% and 7.2%, respectively (FIG. 4d).

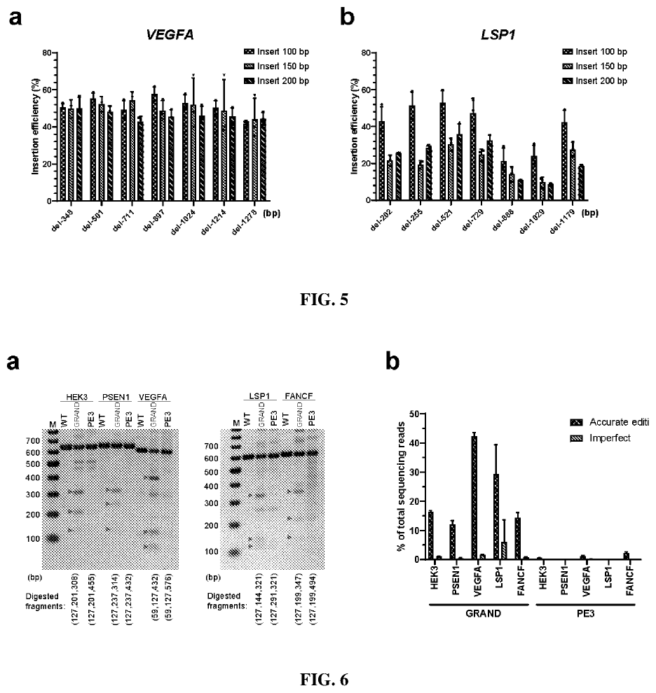

GRAND editing allows insertion of large fragment and meanwhile deletion of the sequences between two nicks. We explored whether GRAND editing could insert large fragment and generate large deletion. Fourteen pairs of pegRNAs were designed to target VEGFA or LSP1 loci for insertion of 100, 150 or 200 bp, and the distances between two pegRNAs ranged from 202 bp to 1278 bp. Most pairs of pegRNAs exhibited comparable insertion efficiencies for each locus, suggesting that distances between paired pegRNAs at least up to ˜1.3 kb may not impede the insertion efficiency (FIG. 5a-b).

We also compared GRAND editing with PE3 that is the standard method for generating insertions using prime editing. While GRAND editing induced 12.0% to 42.4% insertion of 150 bp on five different loci, PE3 induced 0%-2.2% insertion (FIG. 6a-b).

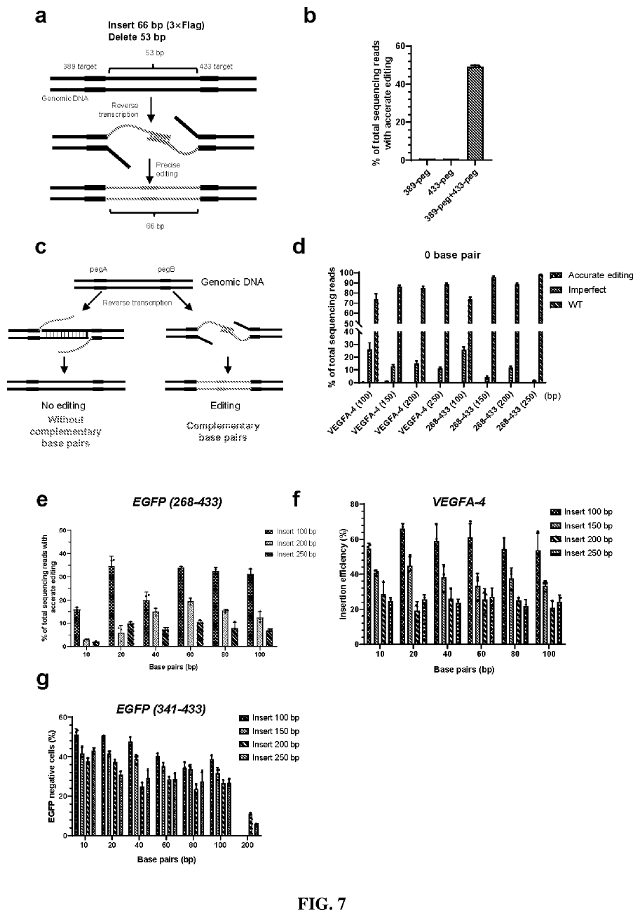

To examine the requirement of paired pegRNAs, each engineered pegRNA was transfected with nCas9-RT, aiming to insert 66 bp of 3×Flag sequence (FIG. 7a). The result showed no editing events by a single pegRNA treatment, while paired pegRNAs exhibited efficient insertion of 66 bp (FIG. 7b). It was not surprising as the ssDNA reverse transcribed from the pegRNAs' RTTs could not hybridize with the genomic sequences to induce 5′ flap, therefore a single pegRNA was not functional.

We then investigated whether the partial complementary sequences between paired pegRNA were required. The paired pegRNA showed no editing when two RTT have no complementary sequences (FIG. 7c-d). In contrast, when two RTTs had 20, 40, 60, 80 or 100 bp complementary sequences to each other, they all exhibited efficient insertion of a 100, 150, 200 or 250 bp sequences for different pairs of pegRNAs (FIG. 7e-g). Interestingly. 10 bp complementary sequence supported efficient insertion for 2 out of 3 pegRNAs pairs (FIG. 7e-g). In contrast, 200 bp complementary sequence dramatically reduced editing efficiency when comparing with 20-100 bp complementary sequences (FIG. 7g).

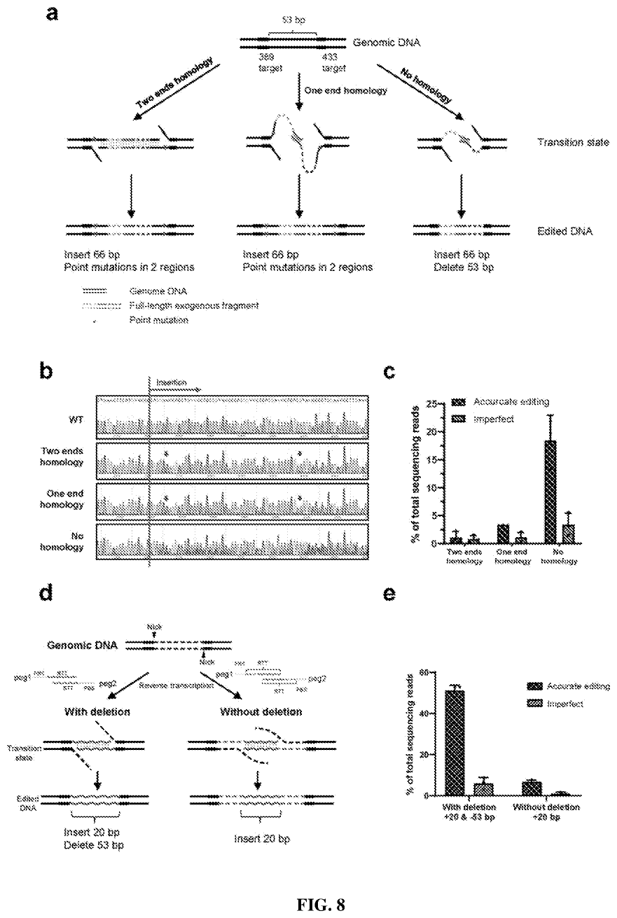

To investigate the role of RTT homology, we designed three pairs of pegRNAs whose RTTs had one end or two ends to be homologous to the target site, or completely no homology (FIG. 8a). All three pairs of pegRNAs had partially complementary RTTs to each other. When both ends of RTTs were homologous to genomic sequences, 1.0% of 66 bp insertion was observed; and 3.3% insertion efficiency was observed when one end of RTTs was homologous to genomic sequences. These efficiencies were significantly lower than the group treated dual pegRNAs with non-homologous RTTs (18.4%) (FIG. 8b-c). Moreover, the first two pairs could effectively install point mutations but not perform targeted insertion of 66 bp, indicating their ability to work as PE when homologous sequences were in RTTs (FIG. 8b). These data suggest that in GRAND editing, the step of hybridization between genomic sequences and ssDNA reverse transcribed from RTT, impedes the insertion process. It is opposite to PE, which requires this hybridization step to resolve 3′ flap.

GRAND editing introduces targeted insertion with deletion of the sequence between two nicks. To understand whether such deletion is preferred, the efficiency of a 20 bp insertion was examined (FIG. 8d). While insertion plus deletion introduced 51.1% editing events, the efficiency of insertion without deletion was 6.7% (FIG. 8e). Insertion without deletion needs homologous sequences in RTT, which causes reduced efficiency of insertion (FIG. 8d-e).

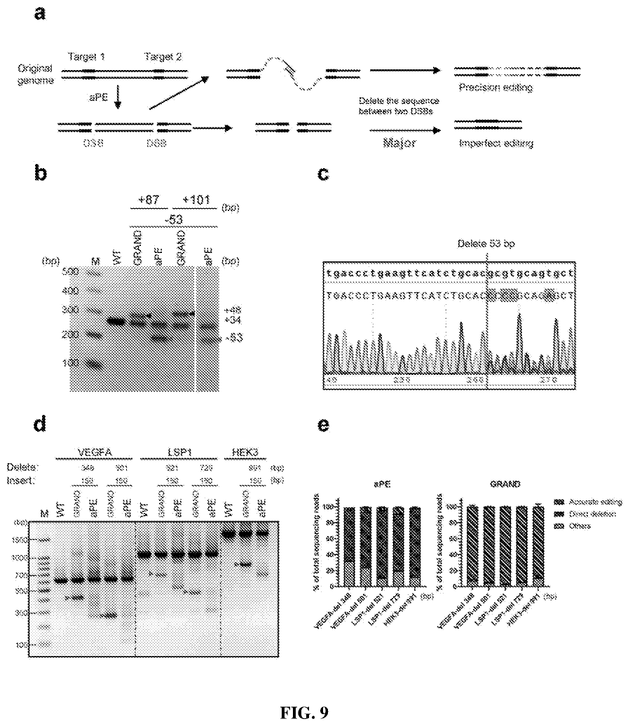

Next, we investigated whether Cas9 nickase in GRAND editing could be replaced by wild type Cas9. Wild type Cas9-mediated GRAND editing (full active Cas9 nuclease-reverse transcriptase, aPE) showed no clear insertions of 87 or 101 bp, and the major outcomes were deletions between the two double stranded breaks (DSBs) (FIG. 9a-c). We further examined 5 pairs of pegRNAs to compare aPE and GRAND editing for insertion of 150 bp. GRAND editing induced efficient insertion, and almost no direct deletion between two nick sites was observed (FIG. 9d-e). In contrast, aPE was not efficient for targeted insertion (FIG. 9d), and the majority of the editing outcomes were deletion between two cutting sites with a small portion of correct insertion (FIG. 9e). These data suggest that the kinetics of repairing DSB is faster than the RT process.

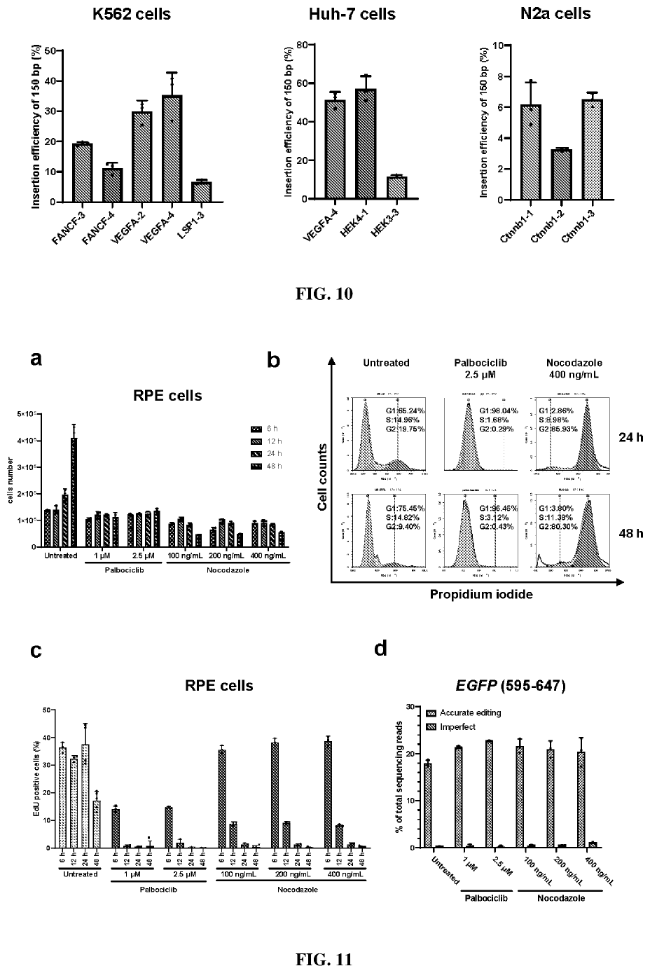

Furthermore, we examined GRAND editing at multiple endogenous sites in three additional cell lines, including human K562 cells, human Huh-7 cells and mouse N2a cells. GRAND editing generates targeted insertion frequencies of 6.5% to 35.2% for K562 cells, 11.5% to 57.0% for Huh-7 cells and 3.3% to 6.5% for N2a cells (FIG. 10).

To determine whether GRAND editing-mediated targeted insertion is cell cycle independent, we used small molecule drugs to arrest the cell cycle of human retinal pigment epithelium (RPE) cell line. Palbociclib, a Cdk4 and Cdk6 inhibitor, effectively arrests cells in G1 phase. Nocodazole is the microtubule-depolymerizing drug to block cells in G2/M. With 1 or 2.5 μM Palbociclib or 100-400 ng/ml Nocodazole treatment, growth of RPE cells was fully inhibited (FIG. 11a). Flow cytometry analysis showed that Palbociclib treated RPE cells were fully arrested at G1 phase, and Nocodazole treatment led to arrest at G2 phase (FIG. 11b). In support, DNA synthesis analysis via 5-ethynyl-2′-deoxyuridine (EdU) incorporation showed that Palbociclib or Nocodazole treatment significantly suppressed global DNA replication at 6 or 12 hours, and near fully inhibited replication at 12-48 or 24-48 hours, respectively (FIG. 11c). Collectively, these data indicate that Palbociclib or Nocodazole treatment successfully arrest RPE cells in G1 or G2 phase (FIG. 11a-c). Next, we conducted the GRAND editing on RPE cells with Palbociclib or Nocodazole treatment. RPE cells with each drug treatment had comparable editing as the untreated cells, indicating GRAND editing is cell-cycle independent (FIG. 11d).

PE editing uses a homologous RTT to target region with desired edits, thus 3′ Flap containing edits hybridized with genomic sequences to form 5′ Flap via Flap equilibration process. Then, the 5′ flap is cleavage and 3′ flap ligation is performed. In contrast, if the RTT show no sequence similarity to the target region, it cannot hybridize with the genomic sequences, thus no 5′ Flap can form. Our data showed that using a single pegRNA of Grand editing generated no editing events, confirming that PE but not Grand Editing requires a homologous RTT to hybridize with the target sequences (FIG. 7b).

For the first time, we demonstrated the feasibility of using a pair of pegRNA can site-specifically and efficiently induce large insertion (ranged from 20-˜1000 bp) (FIG. 1). This length of insertion in our study is beyond the scope of PE editing. We contemplated that the high efficiency of large fragment insertion may due to two processes of Grand editing that are distinct from original PE system: 1) the complementary of two 3′ Flap allowed hybridization to each other to form double-stranded DNA, to prevent cleavage by structure-specific endonucleases; 2) It is possible that GAP filling machinery for both strand facilitates forming desired 5′ Flaps; 3) Grand editing may not require DNA repair machinery to use edited DNA as the template to eliminate the unedited strand.

Grand editing introduces large insertion accompanied by a small or large precise deletion between two nicks. It is particularly suitable for insertion of the desired sequences (e.g. an exon) into the intron region and meanwhile deletion of the faulty sequences to correct various SNPs using one treatment. We expect that Grand editing expand the scope of precise editing from editing one to dozens base pairs to exon installation. We applied Grand editing to install a bsd gene or repair a “broken” EGFP gene into the genome and demonstrated its full activity (FIG. 3). Moreover, about 14% of human pathogenic mutations are duplication and deletion/insertion, which is also could be corrected by Grand editing.

### Example 2. Improved pegRNA Structures

In this example, we tested three modified pegRNA structures and showed that they improved the efficiency of prime editing and Grand editing.

A first design is illustrated in FIG. 12, in which a tail is introduced to the 3′ end of the pegRNA that is able to form a hairpin with PBS or the RT template (FIG. 12a). The editing efficiency of such a modified pegRNA (hp-pegRNA) was compared to a reference wt-pegRNA in HEK293T-eGFP cells targeting the eGFP gene. As shown in FIG. 12b-c, the hp-pegRNA (R5-R) had higher editing efficiency in 10 endogenous gene loci in HEK293T cells and N2a cells compared with WT-pegRNA.

It is contemplated that the hairpin that involves the PBS reduces the interaction between the PBS and the complementary guide sequence (spacer), ensuring that the guide sequence functions effectively to bind to the target editing site. Also, the ensuing stabilized pegRNA can more readily assemble with the Cas9-RT enzyme.

A second design is illustrated in FIG. 13, in which a poly(A) tail is appended at the 3′ end of a conventional pegRNA (FIG. 13a). In the testing, pegRNA with 100-nt RT included 4 mutations in 89-nt editing window were prepared. Sanger sequencing results compared the editing efficiency of PE2 or PE3 system with or without poly-A tail element. Likewise, pegRNA with 200-nt RT included 6 mutations in 190-nt editing window were tested. Sanger sequencing results show that combining PE3 with Poly-A tail element greatly increased the editing efficiency (FIG. 13b-d).

It is contemplated that the addition of the poly(A) tail improved the stability of the pegRNA, leading to improved editing.

A third design is illustrated in FIG. 14, in which a tail is introduced to the 3′ end of the pegRNA that is able to form a loop by binding to a portion of the RT template, or the sgRNA (e.g., the scaffold). The modified pegRNA was used to insert different lengths of fragments using the Grand editing system to disrupt the expression of EGFP by gene insertion. In FIG. 14b left panel, a representative flow cytometry analysis shows the different editing efficiency with or without the structure loop (SL). As summarized in the left panel, the introduction of the SL significantly improved the Grand editing efficiency in all situations.

It is contemplated that the structure loop both stabilizes the pegRNA and reduces the interaction between the PBS and the complementary guide sequence (spacer). Like the hairpin in the first design, such a structure facilitates loading the pegRNA to the Cas9-RT enzyme and enables the guide sequence to function more effectively to bind to the target editing site.

These improved pegRNA structures can be used for both the conventional prime editing systems and in the currently disclosed Grand editing systems, without limitation.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. A method for introducing a nucleic acid sequence into a target DNA sequence at a target site, comprising contacting the target DNA sequence with
(a) a Cas protein and a reverse transcriptase,
(b) a first prime editing guide RNA (pegRNA) comprising a first CRISPR RNA (crRNA), and a first reverse transcriptase (RT) template sequence, and
(c) a second prime editing guide RNA (pegRNA) comprising a second crRNA, and a second RT template sequence,
wherein (i) the first RT template sequence comprises a first fragment and a first pairing fragment, (ii) the second RT template sequence comprises a second fragment and a second pairing fragment, (iii) the first pairing fragment and the second pairing fragment are complementary to each other, (iv) the first fragment and the second fragment each has a length of 0-2000 nt, and (v) the first fragment, the first pairing fragment, and a reverse-complement of the second fragment collectively encode one of the strands of the nucleic acid sequence.

2. The method of claim 1, wherein the first pegRNA further comprises a first primer-binding site (PBS) and a first spacer, enabling the reverse transcriptase to reverse-transcribe the first template sequence at a first PBS target sequence near the target site that is complementary to the first PBS, and wherein the second pegRNA further comprises a second PBS and a second spacer, enabling the reverse transcriptase to reverse-transcribe the second template sequence at a second PBS target sequence near the target site that is complementary to the second PBS.

3. The method of claim 2, wherein the Cas protein is a nickase.

4. The method of claim 3, wherein each pegRNA includes the first or second crRNA, the first or second pairing fragment, the first or second fragment, and the first or second PBS from 5′ to 3′ orientation.

5. The method of claim 2, wherein the Cas protein is a Cas12 protein.

6. The method of claim 5, wherein each pegRNA includes the first or second crRNA, the first or second PBS, the first or second fragment, and the first or second pairing fragment, from 3′ to 5′ orientation.

7. The method of claim 2, wherein the reverse transcription of the first RT template sequence and the second RT template sequence results in pairing of the reverse-transcribed first pairing fragment and the reverse-transcribed second pairing fragment.

8. The method of claim 7, wherein the contacting occurs in the presence of a DNA repair system, which forms a double-stranded DNA sequence introduced at the target site, wherein one strand of the double-stranded DNA sequence is encoded by the first fragment, the first pairing fragment, and a reverse-complement of the second fragment collectively.

9-11. (canceled)

12. The method of claim 1, wherein the first fragment and the second fragment each independently has less than 95% sequence complementarity to the target DNA.

13. The method of claim 2, wherein the first pegRNA or the second pegRNA further comprises a tail that (a) is able to form a hairpin or loop with itself, the PBS, the RT template sequence, the crRNA, or a combination thereof, or (b) comprises a poly(A), poly(U) or poly(C) sequence, or an RNA binding domain.

14-17. (canceled)

18. The method of claim 1, wherein the reverse transcriptase is M-MLV reverse transcriptase or a reverse transcriptase that can function under physiological conditions.

19-20. (canceled)

21. A method for introducing a nucleic acid sequence into a target DNA sequence at a target site, comprising contacting the target DNA sequence with
(a) a Cas protein and a reverse transcriptase,
(b) a first prime editing guide RNA (pegRNA) comprising a first crRNA, and a first reverse transcriptase (RT) template sequence,
(c) a second prime editing guide RNA (pegRNA) comprising a second crRNA, and a second RT template sequence, and
(d) a partially double-stranded DNA comprising a first single-stranded portion, a duplex portion, and a second single-stranded portion,
wherein (i) the first single single-stranded portion has sequence homology to the first RT template sequence, and (ii) the second single-stranded portion has sequence homology to the second RT template sequence.

22-26. (canceled)

27. A prime editing guide RNA (pegRNA) comprising a crRNA, a reverse transcriptase (RT) template sequence, a primer-binding site (PBS), and a tail at the 3′ side of the PBS, wherein the tail (a) is able to form a hairpin, a loop or a complex structural form with itself, the PBS, the RT template sequence, the crRNA, or a combination thereof, or (b) comprises a poly(A), poly(C), or poly(U) tail, or poly(G) sequence, or a structure/sequence recognized by RNA binding proteins.

28. A method of conducting genome editing in a cell, comprising contacting the genomic DNA of the cell with a pegRNA of claim 27, a Cas protein and a reverse transcriptase.

29-31. (canceled)
